(19) United States
(12) Patent Application Publication
(10) Pub. No.: US 2026/0199479 A1
(43) Pub. Date: Jul. 16, 2026

(54) DRUG LINKER AND LINKER-CONJUGATED COMPOUND

(71) Applicant: Anti-Microbial Savior BioteQ Co., Ltd., Kaohsiung City (TW)

(72) Inventors: Yi-Ju Tsai, Kaohsiung City (TW); Guo-Rong Luo, Kaohsiung City (TW); Ying-Ting Yeh, Kaohsiung City (TW)

(73) Assignee: Anti-Microbial Savior BioteQ Co., Ltd., Kaohsiung City (TW)

(21) Appl. No.: 19/136,290

(22) PCT Filed: Dec. 18, 2023
(86) PCT No.: PCT/CN2023/139603

§ 371 Date: Jun. 6, 2025

Related U.S. Application Data
(60) Provisional application No. 63/476,157, filed on Dec. 19, 2022.

(51) Int. Cl. A61K 47/54

(52) U.S. Cl. CPC A61K 47/542

(57) ABSTRACT

The present invention provides a drug linker and a linker-conjugated compound. The drug linker may provide an appropriate space, a flexible synthesis site, and a carboxylic acid moiety, and may be used to connect multiple pharmacophores to enhance biological efficacy and drug similarity, and the linker-conjugated compound has good water solubility.

### TECHNICAL FIELD

The present invention relates to a drug linker and a linker-conjugated compound, in which the linker provides a hydroxy group and a carboxyl group moiety as pharmacophore linking points, and also relates to a drug linker and a linker-conjugated compound using a carboxylic acid moiety.

### DESCRIPTION OF RELATED ART

In the field of drug development technology, small molecule drugs play an important role and may be used to design highly specific drugs for various diseases. However, during the treatment process, challenges such as drug resistance and complications arise, resulting in poor therapeutic effects of single-functional drugs. Therefore, new technologies are needed to solve these problems.

Linkers have many benefits in drug treatment and may be used to combine multiple pharmacophores at the molecular level. Whether they are small molecules or monoclonal antibodies, treatment may be simplified by replacing multiple drugs with one drug. This not only improves the treating patient's convenience but also effectively reduces the potential drug interactions. In addition, linkers may improve the drug properties, adjusting drug performance to provide flexibility for better therapeutic effects.

On the other hand, many lead compounds (leads) with excellent pharmacological activities often have defects in physical properties and pharmacokinetics, such as poor water solubility, low oral bioavailability, and rapid metabolism. Therefore, direct clinical applications are limited. The carboxylic acid moiety produces intermediate acyl phosphates in a variety of biosynthetic and metabolic pathways, which in turn affects the pharmacological activity and may form carboxylate ions in solution to increase solubility and bioavailability. For the above reasons, the introduction of carboxylic acids is widely used in the field of drug design.

Based on the above, developing a drug linker and a linker-conjugated compound that use a carboxylic acid moiety to enhance biological efficacy and drug similarity and has good water solubility, is an urgent goal for those skilled in the art.

### SUMMARY

The present invention provides a drug linker and a linker-conjugated compound. The linker provides a hydroxy group and a carboxyl group moiety as pharmacophore linking points, and the use of carboxylic acid moiety may effectively enhance biological efficacy and drug similarity and has good water solubility.

The drug linker of the present invention has a chemical structure represented by the following formula (1):

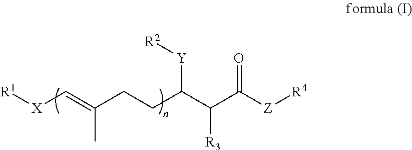

wherein in formula (1),
n is an integer from 1 to 10,
X, Y, and Z are each independently halogen, carbon, oxygen, sulfur, NR⁵, or SiR⁵R⁶,
R¹ is hydrogen, C(═O)R⁵, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, substituted or unsubstituted cyclohexenone, substituted or unsubstituted quinone derivatives, or together with nitrogen forms a substituted or unsubstituted heterocyclic ring having one or more heteroatoms selected from N, O and S,
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S,
R³, R⁵, and R⁶ are each independently hydrogen, substituted or unsubstituted C₁₋₈ alkyl, or substituted or unsubstituted C₅₋₁₀ aryl, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

The linker-conjugated compound of the present invention has a chemical structure represented by the following formula (1-1):

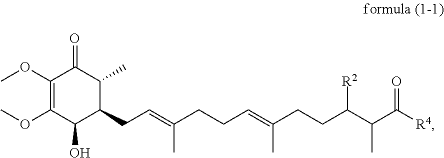

wherein
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

The linker-conjugated compound of the present invention has a chemical structure represented by any one of the following formula (1-2) to formula (1-8):

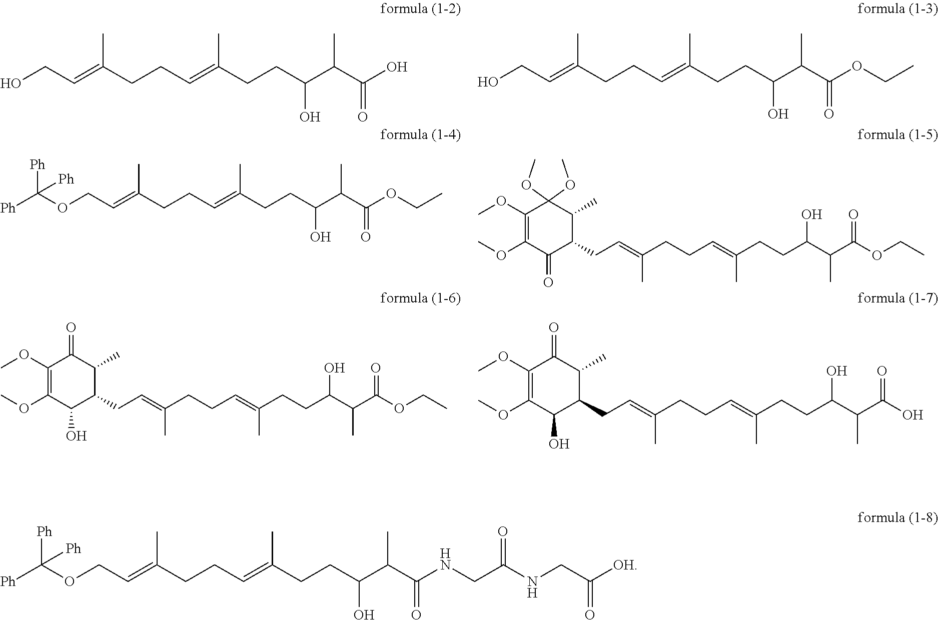

Based on the above, the present invention provides the drug linker and the linker-conjugated compound. The drug linker of the present invention may provide an appropriate space, a flexible synthesis site, and a carboxylic acid moiety, and may be used to connect multiple pharmacophores to enhance biological efficacy and drug similarity, and the linker-conjugated compound has good water solubility.

### DESCRIPTION OF THE EMBODIMENTS

Hereinafter, embodiments of the present invention are described in detail. However, these embodiments are illustrative, and the disclosure of the present invention is not limited thereto.

Herein, a range indicated by “one value to another value” is a general representation which avoids enumerating all values in the range in the specification. Therefore, the record of a specific numerical range covers any number within this numerical range and any smaller numerical range bounded by any number within that numerical range as if such any number and such smaller numerical ranges were expressly written in the specification.

The drug linker of the present invention has a chemical structure represented by the following formula (1):

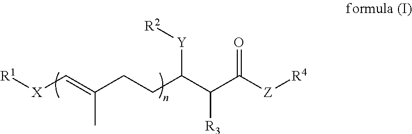

wherein in formula (1),
n is an integer from 1 to 10,
X, Y, and Z are each independently halogen, carbon, oxygen, sulfur, NR⁵, or SiR⁵R⁶,
R¹ is hydrogen, C(═O)R⁵, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, substituted or unsubstituted cyclohexenone, substituted or unsubstituted quinone derivatives, or together with nitrogen forms a substituted or unsubstituted heterocyclic ring having one or more heteroatoms selected from N, O and S,
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S,
R³, R⁵, and R⁶ are each independently hydrogen, substituted or unsubstituted C₁₋₈ alkyl, or substituted or unsubstituted C₅₋₁₀ aryl, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

The linker-conjugated compound of the present invention has a chemical structure represented by the following formula (1-1):

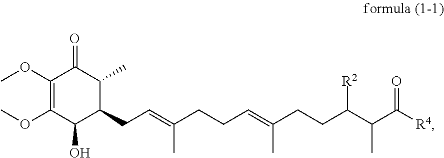

wherein
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

The linker-conjugated compound of the present invention has a chemical structure represented by any one of the following formula (1-2) to formula (1-8):

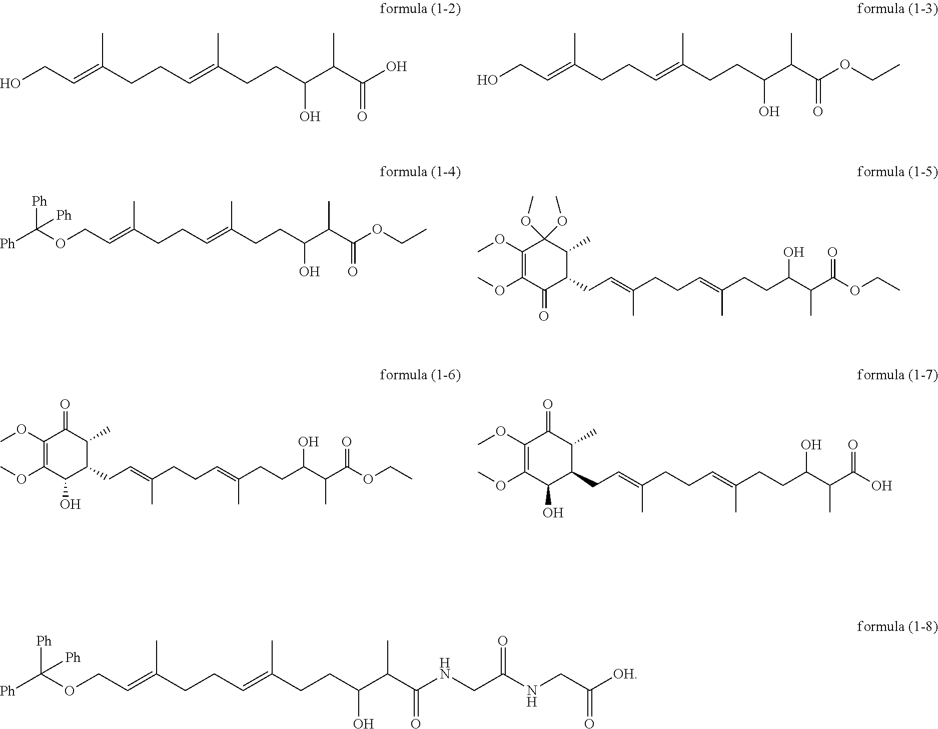

### Preparation Method

All reactions of the present invention were performed in a nitrogen environment using commercial reagents without additional purification. NMR analysis was performed on a Varian Unity 400 MHz spectrometer, using tetramethylsilane (TMS, Merck, Darmstadt, Germany) as internal standard and chloroform-d3 and methanol-d4 as solvents. Chemical shifts are described in (ppm). The splitting pattern is as follows: s=singlet; d=doublet; t=triplet; q=quartet; dd=double doublet; m=multiplet. Analytical thin layer chromatography (TLC) was performed with Art. 5554 Kieselgel 60 GF254 produced by E. Merck (Merck, Darmstadt, Germany). Spots of the compounds were checked using UV indicators irradiated at 254 nm and 366 nm. Column chromatography was prepared using Art. 7734 Kieselgel 60 GF254 (70-400 mesh, Merck, Darmstadt, Germany), produced by E. Merck. PuriFlash® MS (Interchim, Montlucon, France) liquid chromatography electrospray ionization mass spectrometry was used to record the mass spectra.

Abbreviation: Vol. (Volume of solvent (mL)/limiting reactant (g)); THF (tetrahydrofuran); LDA (lithium diisopropylamide); EA (ethyl acetate); TMEDA (tetramethylethylenediamine); LHMDS (lithium bis(trimethylsilyl)amide); n-BuLi (n-butyllithium); DMF (dimethylformamide); DCM (dichloromethane); PPh₃ (triphenyl phosphine); NHS (N-hydroxysuccinimide); EDCI (N-(3-dimethylaminopropyl)-N′-ethylcarbodiimide); and IPA (isopropyl alcohol).

### Synthesis of Linker

The synthesis of the linker is represented by the following reaction formula (1):

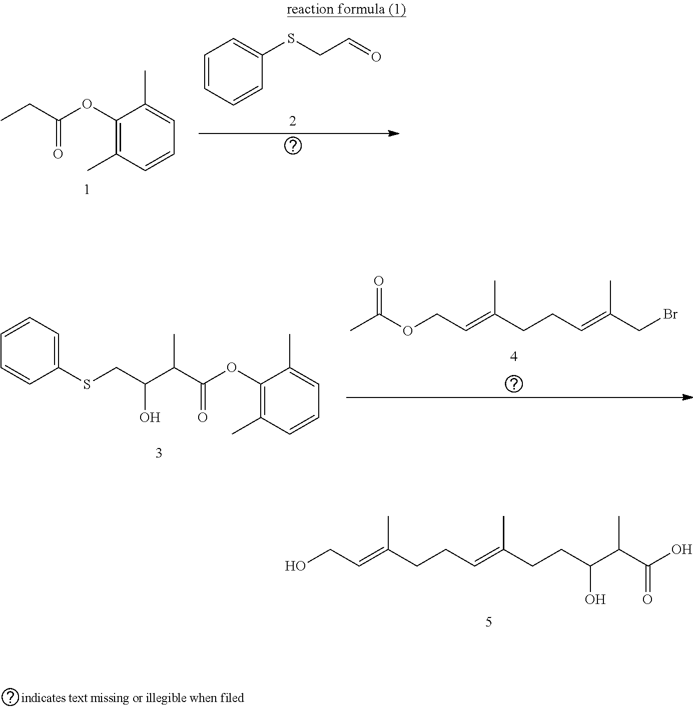

The synthesis of the linker started from 2,6-dimethylphenyl propionate (compound 1). Compound 1 (1.0 eq) and THF (40 Vol.) were slowly added to a stirred solution of THF (130 Vol.) and LDA (2 M in THF, 1.1 eq) at a temperature of −78° C. and stirred at a temperature of −78° C. for 30 minutes. 2-(Phenylthio) acetaldehyde (compound 2) (1.1 eq) and THF (40 Vol.) were slowly added to the mixture, and stirred at a temperature of −78° C. for another 30 minutes. After the reaction was complete (monitored by TLC), the mixture was quenched with 1 M HCl and extracted with EA (2× 400 Vol.). The organic layer was collected, dried over MgSO₄, and concentrated in vacuo. Column chromatography (SiO₂, 1:9 EA/hexane) was used to obtain 2,6-dimethylphenyl 3-hydroxy-2-methyl-4-(phenylthio)butyrate (compound 3) as a yellow oil, with a yield of 37%, ¹H NMR (400 MHz, CDCl₃) δ 7.45-7.40 (m, 2H), 7.34-7.21 (m, 3H), 7.06 (s, 3H), 3.96 (ddd, J=8.0, 6.4, 4.4 Hz, 1H), 3.30 (dd, J=13.7, 4.4 Hz, 1H), 3.14-3.06 (m, 2H), 2.14 (s, 6H), 1.43 (d, J=7.3 Hz, 3H). Chemical formula calculation: C₁₉H₂₂O₃S: m/z 330.1, found value 69.0 (M+K⁺).

Compound 3 (1.1 eq) was then stirred with TMEDA (1.1 eq) in THF (125 Vol.) at a temperature of −78° C. n-BuLi (1.6 M, 1.2 eq) was added to the mixture and stirred at a temperature of −78° C. for 1 h. A solution of (2E,6E)-8-Bromo-3,7-dimethylocta-2,6-dien-1-yl acetate (compound 4) (1.0 eq) in THF (25 Vol.) was added to the mixture, and was stirred at a temperature of −78° C. for another 1 hour. The mixture was quenched with a saturated NH₄Cl aqueous solution and extracted with EA (2×250 Vol.). The organic layer was dried over MgSO₄ and concentrated in vacuo. A yellow oily intermediate was obtained, which could be used in the next step of the synthesis process without purification. The above intermediate dissolved in diethyl ether (20 Vol.) was added to a solution of Li (6.5 eq) in diethylamine (5.0 Vol.) and liquid NH₃ (50 Vol.) and stirred at a temperature of −60° C. for 30 minutes. After the reaction was completed (monitored by TLC), the mixture was diluted with diethyl ether (50 Vol.) and a saturated NH₄Cl aqueous solution (130 Vol.) was slowly added. The organic layer was collected, dried over MgSO₄, and concentrated in vacuo. Column chromatography (SiO₂, 7:3 EA/hexane) provided (6E,10E)-3,12-dihydroxy-2,6,10-trimethyldodec-6,10-dienoic acid (compound 5) as a colorless oil, with a yield of 39%, ¹H NMR (600 MHz, CD₃OD) δ 5.39-5.33 (m, 1H), 5.21-5.15 (m, 1H), 4.08 (d, J=6.8 Hz, 2H), 3.76-3.67 (m, 1H), 2.55-2.38 (m, 1H), 2.22-2.10 (m, 3H), 2.09-2.00 (m, 3H), 1.67 (s, 3H), 1.61 (s, 3H), 1.50-1.42 (m, 1H), 1.19-1.10 (m, 3H). Chemical formula calculation: C₁₅H₂₆O₄: m/z 270.2, found value 291.2 (M+Na−2H⁻), 305.2 (M+Cl⁻).

### Carboxyl Esterification of Linker and Primary Alcohol Substitution

The esterification reaction of the linker is represented by the following reaction formula (2):

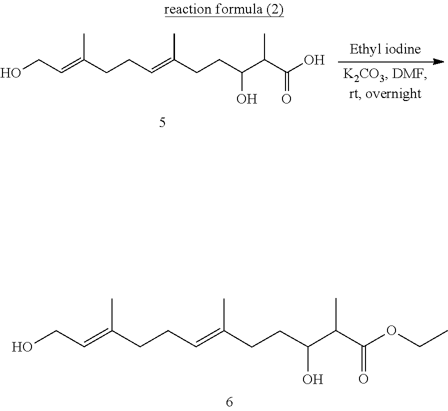 [SMILES: C/C(=C\CO)CC/C=C(\C)CCC(O)C(C)C(=O)O ; CCOC(=O)C(C)C(O)CC/C(C)=C/CC/C(C)=C/CO]

Carboxyl substitution: Ethyl iodide (1.1 eq) was added to a mixture of compound 5 (1.0 eq), K₂CO₃ (3.0 eq), and DMF (20 Vol.) at room temperature and stirred overnight. After the reaction was complete (monitored by TLC), the mixture was quenched with water, and then extracted with EA (2×130 Vol.). The organic layer was collected, dried over MgSO₄, and concentrated in vacuo. Column chromatography (SiO₂, 5:5 EA/hexane) provided (6E,10E)-3,12-Dihydroxy-2,6,10-trimethyldodeca-6,10-dienoic acid ethyl ester as a colorless oil (compound 6), with a yield of 92%, ¹H NMR (400 MHz, CDCl₃) δ 5.44-5.35 (m, 1H), 5.19-5.12 (m, 1H), 4.25-4.07 (m, 4H), 3.90-3.57 (m, 1H), 2.67 (s, 1H), 2.56-2.45 (m, 2H), 2.21-2.00 (m, 6H), 1.66 (s, 3H), 1.60 (s, 3H), 1.54-1.44 (m, 2H), 1.27 (t, J=7.1 Hz, 3H), 1.22-1.15 (m, 3H). Chemical formula calculation: C₁₇H₃₀O₄: m/z 298.2, found value 321.2 (M+Na⁺).

The substitution reaction of the linker is represented by the following reaction formula (3):

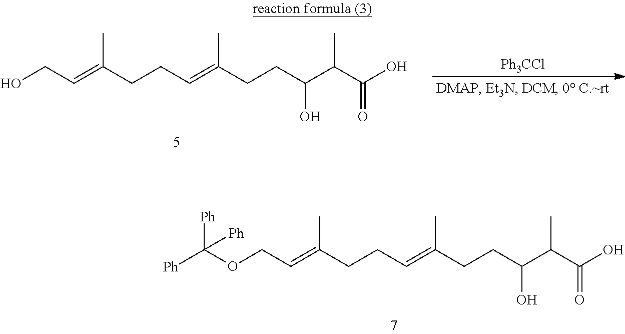 [SMILES: C/C(=C\CO)CC/C=C(\C)CCC(O)C(C)C(=O)O ; "Ph3CCl" ; C/C(=C\COC(c1ccccc1)(c1ccccc1)c1ccccc1)CC/C=C(\C)CCC(O)C(C)C(=O)O]

Primary alcohol substitution: Triphenylmethyl chloride (1.1 eq), 4-dimethylaminopyridine (DMAP, 1.1 eq), triethylamine (1.1 eq), and compound 5 (1 eq) were dissolved in DCM (35 Vol.) and stirred overnight at a temperature from 0° C. to room temperature. After the reaction was complete (monitored by TLC), the solvent was evaporated in vacuo. Column chromatography (SiO₂, 1:9 EA/hexane) was used to obtain (6E,10E)-3-Hydroxy-2,6,10-trimethyl-12-(trityloxy) dodeca-6,10-dienoic acid as a colorless oil (compound 7), with a yield of 55%, ¹H NMR (600 MHz, CDCl₃) δ 7.48-7.44 (m, 6H), 7.32-7.27 (m, 6H), 7.24-7.20 (m, 3H), 5.47-5.40 (m, 1H), 5.22-5.16 (m, 1H), 3.71-3.66 (m, 1H), 3.61 (d, J=6.4 Hz, 2H), 2.59-2.51 (m, 1H), 2.22-2.00 (m, 6H), 1.62 (s, 3H), 1.61-1.52 (m, 2H), 1.46 (s, 3H), 1.24-1.16 (m, 3H). Chemical formula calculation: C₃₄H₄₀O₄: m/z 512.3, found value 511.2 (M−H⁻).

### Conjugation of Active Compound to Linker

The synthesis of the linker-conjugated compound is represented by the following reaction formula (4):

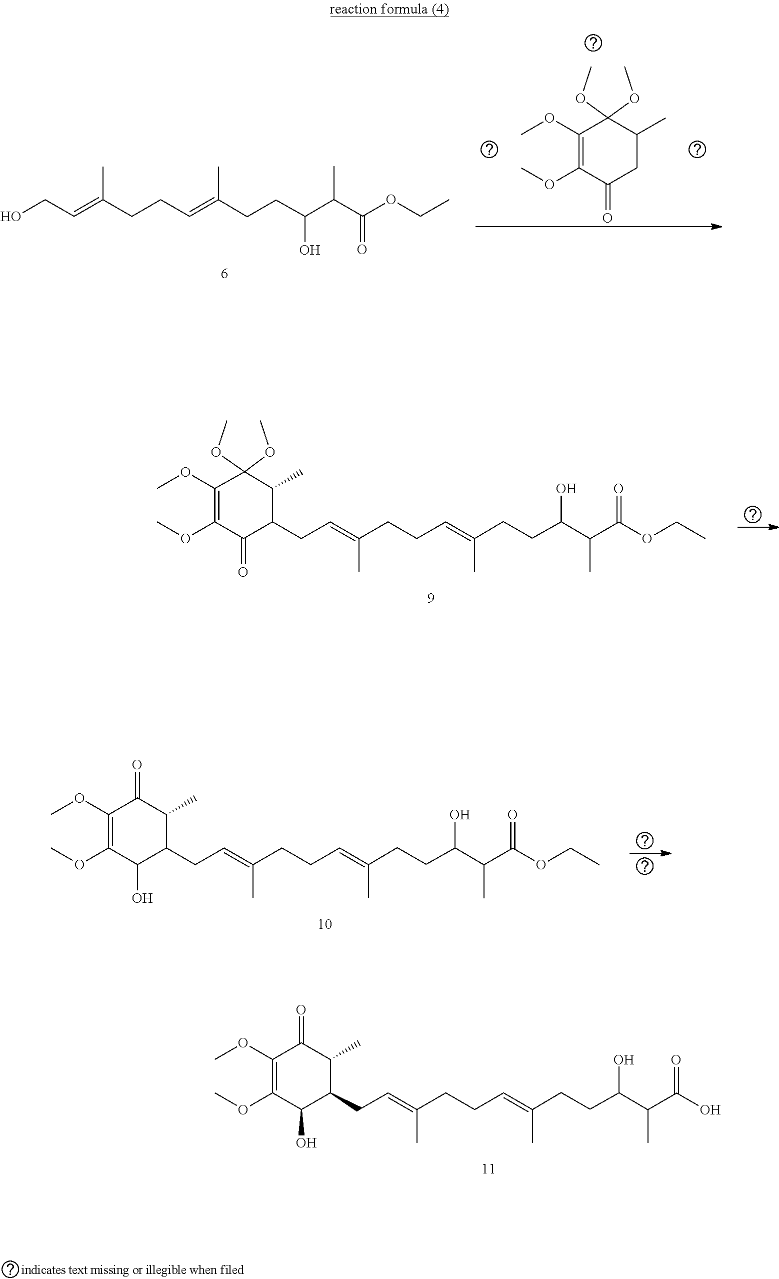

To synthesize the linker-conjugated compound 11, triphenyl phosphine (1.1 eq), tetrabromomethane (1.1 eq), and compound 6 (1 eq) were dissolved in DCM (35 Vol.) and stirred at a temperature of 0° C. After 2 hours, the solvent was filtered and evaporated in vacuo. Then, LHMDS (1 M in THF, 2 eq) was slowly added to the mixture with THF (15 Vol.). The mixture was stirred at a temperature of −78° C. for 2 hours. Compound 8 (1.2 eq) was added to the mixture and stirred at room temperature overnight. After the reaction was completed (monitored by TLC), the mixture was quenched with a saturated NH₄Cl aqueous solution and extracted with EA (3×90 Vol.). The organic layer was collected, dried over MgSO₄, and concentrated in vacuo. Column chromatography (SiO₂, 1:9 EA/hexane) provided ethyl (6E,10E)-3-hydroxy-2,6,10-trimethyl-12-((1S,6R)-3,4,5,5-tetramethoxy-6-methyl-2-oxocyclohex-3-en-1-yl) dodeca-6,10-dienoate (compound 9) as a light yellow oil, with a yield of 30%, ¹H NMR (600 MHz, CDCl₃) δ 5.15 (t, J=6.9 Hz, 1H), 5.07 (t, J=6.9 Hz, 1H), 4.19-4.14 (m, 2H), 4.12 (s, 3H), 3.67 (s, 3H), 3.64 (s, 1H), 3.31 (s, 3H), 3.26 (s, 3H), 2.66-2.55 (m, 2H), 2.54-2.46 (m, 1H), 2.38-2.28 (m, 2H), 2.11-1.96 (m, 6H), 1.63 (s, 3H), 1.59 (s, 3H), 1.27 (t, J=7.1 Hz, 3H), 1.21-1.17 (m, 3H), 0.98 (d, J=6.7 Hz, 3H). Chemical formula calculation: C₂₈H₄₆O₈: m/z 510.3, found value 533.4 (M+Na⁺).

Lithium tri-sec-butylborohydride (1 M in THF, 8.0 eq) was added dropwise to a solution of compound 9 (1.0 eq) in THF (200 Vol.) at a temperature of −70° C. and stirred for 4 h. The reaction was diluted with DCM (500 Vol.) and silica gel was added and stirred for 10 minutes. After the reaction was completed (monitored by TLC), the silica gel was filtered off and the residue was concentrated in vacuo. Column chromatography (SiO₂, 1:9 EA/hexane) provided Ethyl (6E,10E)-3-hydroxy-12-((1S,2S,6R)-2-hydroxy-3,4-dimethoxy-6-methyl-5-oxocyclohex-3-en-1-yl)-2,6,10-trimethyl dodeca-6,10-dienoate (compound 10) as a light yellow oil, with a yield of 30%, ¹H NMR (400 MHz, CDCl₃) δ 5.11-5.16 (m, 2H), 4.26-4.31 (m, 1H), 4.17 (q, J=7.2 Hz, 2H), 4.12 (s, 3H), 3.67 (s, 3H), 3.62-3.64 (m, 1H), 2.50-2.58 (m, 2H), 2.05-2.31 (m, 8H), 1.81-1.87 (m, 1H), 1.65 (s, 3H), 1.62-1.64 (m, 2H), 1.60 (s, 3H), 1.20-1.28 (m, 9H). Chemical formula calculation: C₂₆H₄₂O₇: m/z 466.3, found value 489.4 (M+Na⁺).

NaOH (10 wt % aqueous solution, 10 Vol.) was added dropwise to a solution of compound 10 (1.0 eq) in MeOH (40 Vol.), and stirred at room temperature for 1 hour. After the reaction was completed (monitored by TLC), the reaction mixture was washed with DCM (2×200 Vol.). The aqueous layer was acidified with 1 M HCl and extracted with DCM (2×200 Vol.). The organic layer was collected, dried with MgSO₄, and concentrated in vacuo to obtain compound 11 as a light yellow oil with a yield of 61%, ¹H NMR (400 MHz, CDCl₃) δ 5.12-5.19 (m, 2H), 4.33 (d, J=6.4 Hz, 3H), 4.08 (s, 3H), 3.59 (s, 3H), 3.44-3.48 (m, 1H), 2.37-2.46 (m, 1H), 2.24-2.36 (m, 2H), 1.95-2.21 (m, 7H), 1.76-1.87 (m, 1H), 1.61 (d, J=8.8 Hz, 6H), 1.23 (d, J=7.2 Hz, 3H), 1.18 (d, J=7.2 Hz, 3H). Chemical formula calculation: C₂₄H₃₈O₇: m/z 438.3, found value 437.4 (M−H⁻).

### Conjugation of Amino Acid to Linker

The synthesis of the linker-conjugated amino acid derivatives is represented by the following reaction formula (5):

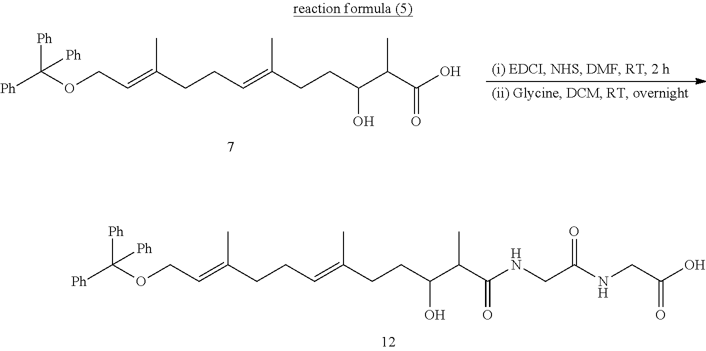 [SMILES: C/C(=C\COC(c1ccccc1)(c1ccccc1)c1ccccc1)CC/C=C(\C)CCC(O)C(C)C(=O)O ; C/C(=C\COC(c1ccccc1)(c1ccccc1)c1ccccc1)CC/C=C(\C)CCC(O)C(C)C(=O)NCC(=O)NCC(=O)O]

EDCI (2.0 eq) was added to a stirred solution of compound 7 (1.0 eq) and NHS (2.0 eq) in DMF (20 Vol.) at room temperature, and the mixture was stirred at room temperature for 2 h. The mixture was extracted with EA (3×130 Vol.). The organic layer was dried over MgSO₄ and concentrated under reduced pressure. The crude compound was dissolved in DCM (20 Vol.), glycine (2.0 eq) was added, and the mixture was stirred at room temperature overnight. The mixture was extracted with DCM (2×100 Vol.). The organic layer was dried over MgSO₄ and concentrated under reduced pressure. Compound 12 was obtained as a light yellow oil (23%) using column chromatography (SiO₂, 100/0 to 50/50 EA/MeOH). ¹H NMR (600 MHz, CDCl₃) δ 7.48-7.44 (m, 6H), 7.31-7.27 (m, 6H), 7.24-7.20 (m, 3H), 5.20-5.14 (m, 1H), 5.14-5.07 (m, 1H), 5.01 (dt, J=2.1, 1.6 Hz, 1H), 4.98 (dt, J=2.1, 1.6 Hz, 1H), 4.94 (dt, J=2.3, 1.2 Hz, 1H), 4.92 (dt, J=2.3, 1.2 Hz, 1H), 3.86-3.80 (m, 1H), 3.60 (d, J=6.3 Hz, 2H), 2.73-2.67 (m, 1H), 2.12-1.99 (m, 6H), 1.76-1.69 (m, 2H), 1.46 (s, 3H), 1.43 (s, 3H), 1.30 (d, J=7.1 Hz, 3H). Chemical formula calculation: C₃₈H₄₆N₂O₆: m/z 626.3, found value 668.4 (M+ACN+H⁺)).

In the following, test methods for testing various properties of the drug linker and the linker-conjugated compound of the present invention are listed.

### Water Solubility Test

### High Performance Liquid Chromatography Analysis Method

| Brand | SHIMADZU LC-2030 C | | |
|---|---|---|---|
| Column | YMC-Pack Pro C18 RS 4.6 × 250 mm | | |
| Protective column | SL12S05 10 × 4.0 mm, S-5 μm, 8 nm | | |
| Flow rate | 1 mL/min | | |
| Column oven | 35° C. | | |
| temperature | | | |
| Sample tray | 15° C. | | |
| temperature | | | |
| Wavelength | 220 nm | | |
| Injection volume | 50 μL | | |
| | Time | Mobile phase: ACN | Mobile phase: 0.1% |
| | (min) | (%) | HCOOH in water (%) |
| Gradient | 0 | 95 | 5 |
| | 20 | 95 | 5 |

### Standard Solution Calibration Line Preparation Method

5 mg of the standard test compound was weighed and dissolved in 5 mL of IPA as a stock solution (1 mg/mL). Double distilled water was then diluted to prepare 1000, 100, 50, 25, 12.5, 10, 5, 2.5, 1.25, 1, 0.5, 0.25, 0.125 μg/mL standard solution calibration lines. 60 μL of each concentration standard solution was used for HPLC analysis. Peak areas were integrated to establish a calibration line, and the accuracy of the analysis method was evaluated through the correlation coefficient (R-squared).

### Water Solubility Inspection Method

1 mL of double distilled water was weighed into a microcentrifuge tube and the test compound was dissolved. Undissolved compounds were ensured to be still present by vortexing for 5 min and sonicating for 20 min. After centrifugation, the supernatant was filtered through a 0.22 μm PTEF membrane. The residue was analyzed by HPLC and the water solubility was calculated from the peak area.

### Cytotoxicity Screening

### Cell Culture

NOZ cells, human gallbladder cancer cells, were obtained from the Japanese Collection of Research Bioresources Cell Bank (JCRB, Japan). Cells were cultured in 90% William's E culture medium (Gibco, USA), 10% fetal bovine serum (FBS) (Gibco, USA), and 1% antibiotics (1×penicillin, streptomycin and glutamine) (Gibco, USA).

HuCCT1 cells, human cholangiocarcinoma cells, were obtained from iCell Bioscience Inc. (iCell, China). Cells were cultured in 90% RPMI 1640 culture medium (Gibco, USA), 10% fetal bovine serum (FBS) (Gibco, USA), and 1% antibiotics (1×penicillin, streptomycin and glutamine) (Gibco, USA).

HuH28 cells, human liver, and cholangiocarcinoma cells, were obtained from the Japanese Collection of Research Bioresources Cell Bank (JCRB, Japan). Cells were cultured in 90% RPMI 1640 culture medium (Gibco, USA), 10% fetal bovine serum (FBS) (Gibco, USA), and 1% antibiotics (1×penicillin, streptomycin and glutamine) (Gibco, USA).

AML12 cells, alpha mouse liver 12, were obtained from the American Type Culture Collection (ATCC, USA). Cells were cultured in DMEM: F12 culture medium (Gibco, USA), 10% fetal bovine serum (FBS) (Gibco, USA), 10 μg/mL insulin, 5.5 μg/mL transferrin, 5 ng/ml selenium, 40 ng/ml dexamethasone (Gibco, USA), and 1% antibiotics (1×penicillin, streptomycin and glutamic acid) (Gibco, USA).

AsPC-1, human pancreatic adenocarcinoma ascites metastatic cells, were obtained from the Bioresource Collection and Research Center (BCRC, Taiwan, China). Cells were cultured in RPMI 1640 culture medium (Gibco, USA) containing 10% FBS (Gibco, USA) and 1% antibiotics (1×penicillin, streptomycin and glutamic acid) (Gibco, USA).

PANC-1, human pancreatic epithelioid carcinoma, was obtained from the American Type Culture Collection (ATCC, USA). Cells were cultured in DMEM culture medium (Gibco, USA) containing 10% FBS (Gibco, USA) and 1% antibiotics (1×penicillin, streptomycin and glutamic acid) (Gibco, USA).

BxPC-3, human pancreatic adenocarcinoma cells, were obtained from the Bioresource Collection and Research Center (BCRC, Taiwan, China). Cells were cultured in RPMI 1640 culture medium (Gibco, USA) containing 10% FBS (Gibco, USA) and 1% antibiotics (1×penicillin, streptomycin and glutamic acid) (Gibco, USA).

A549, human non-small cell lung cancer cells, were obtained from the Bioresource Collection and Research Center (BCRC, Taiwan, China). Cells were cultured in F12K culture medium (Gibco, USA) containing 10% FBS (Gibco, USA) and 1% antibiotics (1×penicillin, streptomycin and glutamic acid) (Gibco, USA).

MCF7, human breast cancer, was obtained from the Bioresource Collection and Research Center (BCRC, Taiwan, China). Cells were cultured in minimal essential Eagle's culture medium (FBS) (Gibco, USA) containing 2 mM L-glutamic acid, 1.5 mg/mL sodium bicarbonate, 0.1 mM non-essential amino acids, 1.0 mM sodium pyruvate (Gibco, USA), 10% fetal bovine serum (Gibco, USA) and 1% antibiotics (1×penicillin, streptomycin and glutamic acid) (Gibco, USA).

HCT-15, colorectal adenocarcinoma, was obtained from the Bioresource Collection and Research Center (BCRC, Taiwan, China). Cells were cultured in RPMI 1640 culture medium (Gibco, USA) containing 10% fetal bovine serum (FBS) (Gibco, USA) and 1% antibiotics (1×penicillin, streptomycin and glutamic acid) (Gibco, USA).

Caco-2, colorectal adenocarcinoma, was obtained from the Bioresource Collection and Research Center (Elabscience, USA). Cells were cultured in minimal essential Eagle's culture medium (FBS) (Gibco, USA) containing 2 mM L-glutamic acid, 1.5 mg/mL sodium bicarbonate, 0.1 mM non-essential amino acids, 1.0 mM sodium pyruvate (Gibco, USA), 20% fetal bovine serum (Gibco, USA), and 1% antibiotics (1×penicillin, streptomycin and glutamic acid) (Gibco, USA).

Vero E6 cells, cercopithecus aethiops renal epithelial cells, were obtained from the American Type Culture Collection (ATCC, USA). Cells were cultured in Dulbecco's Modified Eagle Medium (DMEM) (Gibco, USA) containing 10% FBS (Gibco, USA), 100 U/mL penicillin, and 100 μg/mL streptomycin (Gibco, USA).

### Cell Viability Assessment

Cell viability was assessed with PrestoBlue assay (Invitrogen, USA). Cells were seeded into 96-well plates and cultured with test compounds according to the manufacturer's protocol. After 48 hours of exposure to test compounds, the culture medium was discarded and rinsed with PBS. Then, add 10 μL PrestoBlue reagent containing 90 μL culture medium to the cells and incubate the cell plate at 37° C. in the dark for 120 min. Absorbance was measured at the experimental wavelength of 570 nm and the normalized wavelength of 600 nm in a SpectraMax iD3 microplate reader. The result was corrected by subtracting the blank value. Cell viability was expressed as a percentage relative to the control, and calculated using the following formula: cell viability (%)=[(Absorbance of test wells at 570 nm−Absorbance of only culture medium wells at 570 nm−Absorbance of test wells at 600 nm+Absorbance of only culture medium wells at 600 nm)/Absorbance of the control well at 570 nm−Absorbance of only culture medium wells at 570 nm-Absorbance of the control well at 600 nm+Absorbance nm of only culture medium wells at 600 nm)]×100. Data are expressed as mean±standard deviation as displayed in GraphPad Prism.

In the following, the test results of various properties of the drug linker and the linker-conjugated compound of the present invention are listed.

### Solubilizer Effect Test

At physiological pH 7.4, ionization of carboxylic acids increases their ability to form hydrogen bonds with neighboring water molecules and, therefore, improves their overall water solubility. In order to verify that the drug linker of the present invention may effectively improve the water solubility of the compound, a water solubility test (Table 1) was performed. Both the drug linker and farnesol of the present invention contain a hydroxy group at the 1′ position and 15 carbons in the linear structure. However, compared with farnesol, the drug linker of the present invention adds a hydroxy group at the 10′-position and a carboxyl group at the 12′-position, and the water solubility increases sharply by nearly 9 times, from 87.4 μg/mL to 780.1 μg/mL. The results prove that compounds designed using the drug linker of the present invention may effectively improve water solubility.

### Table 1

### Pharmacophore Linker Effect Test

| Compound | Water solubility (μg/mL) |
|---|---|
| 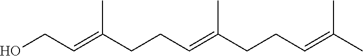 [SMILES: CC(C)=CCC/C(C)=C/CC/C(C)=C/CO] | 87.4 |
| Farnesol | |
| 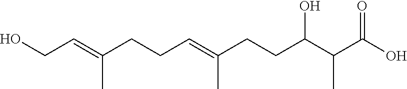 [SMILES: C/C(=C\CO)CC/C=C(\C)CCC(O)C(C)C(=O)O] | 780.1 |
| Drug linker of the present invention | |

Bi/multifunctional drugs connect two or more pharmacophores through linkers to achieve better effects than when used alone. In such a technology, the design of the linker is crucial to maintain the activity of the original drug while avoiding the introduction of additional toxicity. Furthermore, an excellent linker should be synthetically flexible to allow easy conjugation with the pharmacophore.

The drug linker of the present invention has a chemical structure represented by the following formula (1):

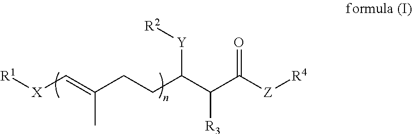

Taking the linker of the present invention (n=2; X, Y, Z=O; R¹, R², R⁴=H; R³=CH3) as an example, the linker has significant advantages in terms of synthetic elastic properties. The alcohol group on R¹—X is a primary alcohol and is prone to substitution reaction (reaction formula (3)). In addition, when the alcohol groups of R¹—X and R²—Y are converted into halides via Appel reaction, the halide intermediate is easily combined with other nucleophiles such as alcohols, organolithium, enols, amines, Thiol reaction (the first step of reaction formula (4)). At the same time, the carboxyl group in R⁴—Z shows high reactivity and may undergo esterification reaction (reaction formula (2)) under acid/base catalysis, or may react with amine moieties on small molecules, amino acids, or protein residues through crosslinking agents to form an amide (reaction formula (5)). The diverse connection methods greatly expand its application scope. Structural flexibility is also a key point of the linker, allowing the entire drug molecule to move freely after binding to the target. A common indicator of molecular flexibility is the number of rotatable bonds (NRot), with higher values indicating better flexibility. According to Table 2 below, the linker of the present invention has a higher NRot value, indicating that it has great potential as a linker in bi/multifunctional drugs. On the other hand, non-cytotoxicity is a basic requirement of the linker. Various cell lines were used to conduct cytotoxicity tests, and cell experiments proved that the linker of the present invention is not cytotoxic to either normal cells or cancer cells (Table 3).

TABLE 2

| Chemical structure | NRot |
|---|---|
| Linker of the present invention | 9 |
| 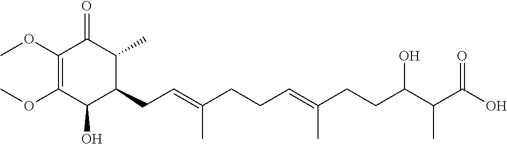 [SMILES: COC1=C(OC)[C@H](O)[C@H](C/C=C(\C)CC/C=C(\C)CCC(O)C(C)C(=O)O)[C@@H](C)C1=O] | 12 |
| Compound 11 | |

TABLE 3
Linker of the present invention

| Cell type | Cell line | 20 μg/mL 48-hour survival rate |
|---|---|---|
| Gallbladder cancer | NOZ | 107.26 ± 14.11% |
| cells | HuCCT1 | 110.04 ± 0.17% |
| | HuH28 | 87.92 ± 5.14% |
| Pancreatic cancer cells | PANC-1 | 106.67 ± 8.24% |
| | BxPC-3 | 91.33 ± 5.93% |
| | AsPC-1 | 93.02 ± 5.01% |
| Colon cancer cells | Caco-2 | 99.94 ± 3.16% |
| | HCT-15 | 88.96 ± 15.45% |
| Breast cancer cells | MCF-7 | 89.45 ± 8.95% |
| Lung cancer cells | A549 | 82.28 ± 5.53% |
| Normal hepatocytes | AML12 | 91.99 ± 2.6% |
| Normal kidney cells | Vero E6 | 96.28 ± 3.66% |

In summary, the present invention provides the drug linker and the linker-conjugated compound. The drug linker of the present invention may provide an appropriate space, a flexible synthesis site, and a carboxylic acid moiety, and may be used to connect multiple pharmacophores to enhance biological efficacy and drug similarity, and the linker-conjugated compound has good water solubility sex. The drug linker of the present invention may be used to enhance solubility and is not cytotoxic.

What is claimed is:

1. A drug linker, characterized in that it has a chemical structure represented by the following formula (1): 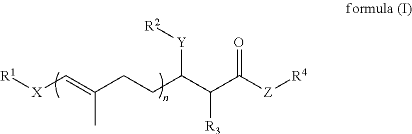
wherein in formula (1),
n is an integer from 1 to 10,
X, Y, and Z are each independently halogen, carbon, oxygen, sulfur, NR⁵, or SiR⁵R⁶,
R¹ is hydrogen, C(═O)R⁵, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, substituted or unsubstituted cyclohexenone, substituted or unsubstituted quinone derivatives, or together with nitrogen forms a substituted or unsubstituted heterocyclic ring having one or more heteroatoms selected from N, O and S,
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S,
R³, R⁵, and R⁶ are each independently hydrogen, substituted or unsubstituted C₁₋₈ alkyl, or substituted or unsubstituted C₅₋₁₀ aryl, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

2. A linker-conjugated compound, characterized in that it has a chemical structure represented by the following formula (1-1): 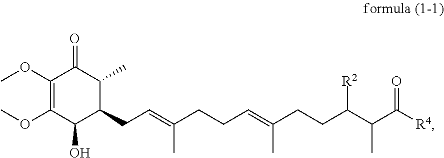
wherein
R² is hydrogen, substituted or unsubstituted C₁₋₈ alkyl, substituted or unsubstituted C₂₋₈ alkenyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S, and
R⁴ is hydrogen, substituted or unsubstituted C₁₋₈ alkyl, substituted or unsubstituted C₅₋₁₀ aryl, substituted or unsubstituted C₃₋₈ cycloalkyl, substituted or unsubstituted C₅₋₁₀ heteroaryl, substituted or unsubstituted C₅₋₁₀ lactone, or together with nitrogen forms a substituted or unsubstituted heterocyclic ring having one or more heteroatoms selected from N, O and S.

3. A linker-conjugated compound, characterized in that it has a chemical structure represented by any one of the following formula (1-2) to formula (1-8): [chemical structure]
